(19) United States
(12) Patent Application Publication
(10) Pub. No.: US 2026/0357180 A1
(43) Pub. Date: Nov. 12, 2026

(54) SYSTEM AND METHOD FOR TEMPERATURE CONTROL OF A CELL STRUCTURE

(71) Applicant: GLOBAL LIFE SCIENCES SOLUTIONS USA LLC, Marlborough, MA (US)

(72) Inventors: Mahesh BHAT, Bengaluru (IN); Ajay GORE, Bengaluru (IN); Sudeep DAM, Bengaluru (IN); Saravanan BALAKRISHNAN, Bengaluru (IN); Kandakumar MURUGESAN, Bengaluru (IN); Nagaraj RAO, Bengaluru (IN); Bhaskaran SAVARIMUTHU, Bengaluru (IN); Santhosh KUMAR, Bengaluru (IN); Suresh PANDI, Bengaluru (IN)

(73) Assignee: GLOBAL LIFE SCIENCES SOLUTIONS USA LLC, Marlborough, MA (US)

(21) Appl. No.: 19/137,136

(22) PCT Filed: Dec. 20, 2023
(86) PCT No.: PCT/US2023/085072

§ 371 Date: Jun. 9, 2025

(30) Foreign Application Priority Data
Jan. 5, 2023 (IN) 202311001132

(51) Int. Cl. C12M 3/06; C12M 1/00; C12M 1/02

(52) U.S. Cl. CPC C12M 27/16; C12M 23/14; C12M 23/26; C12M 41/22

(57) ABSTRACT

System and methods are provided for temperature control of a cell culture or media. The systems and methods include a first enclosure containing a cell culture positioned on a surface area of a platform. The systems and methods include a temperature regulating element, in contact with the first enclosure, through which a heat transfer fluid is circulated. The heat transfer fluid is either heated or cooled, such that heating or cooling is imparted to the first enclosure, thereby heating or cooling the cell culture.

### CROSS-REFERENCE TO RELATED APPLICATIONS

This application is a national stage of International Application No. PCT/US2023/085072 filed on Dec. 20, 2023, which claims the benefit of India Application No. 202311001132, filed on Jan. 5, 2023, all of which are hereby incorporated by reference herein in their entireties.

### FIELD

One or more embodiments of the subject matter described herein generally relates to systems and methods for temperature control of a cell culture or media.

### BACKGROUND

A bioreactor provides a closed-loop, controlled environment to promote growth or cultivation of cells. During the cultivation process, cells are combined with a culture medium or media to form a cell culture within a bag. The bioreactor controls an agitation process, maintains a temperature and pH of the media, gas mixture and flow within the bag, and/or the like for cultivation and growth of the cells over time (e.g., hours, days).

With regard to temperature control, conventional rocking-type bioreactors often implement a heater pad underneath the culture bag to control heating of the contents of the culture bag. Given state of the art design constraints there is no mechanism within the bioreactor to provide cooling. Rather, when cooling is desired (e.g., for virus inactivation, product preservation, etc.) the entire bioreactor is placed within a cold room (e.g., walk-in refrigerator) or the contents of the bioreactor are transferred to a separate container that is subsequently cooled. Additionally, certain therapeutics (e.g., mRNA) are highly sensitive to temperature and degrade at warmer temperatures, necessitation the transition to a cool environment quickly. However, such processes are cumbersome, require additional equipment, and make maintaining sterility more difficult. Additionally, when preparing for product harvest it is often advantageous to simulate large scale production processes (e.g., so that scale up is easier). To simulate such a process a cooling step is necessary prior to filtration (i.e., at large scale a thermal jacket is used to cool the bioreactor contents prior to filtration and harvest). It would be advantageous to provide an improved heating/cooling mechanism that is integrated into the bioreactor.

US20210071123 describes a bioreactor system that can include a cooling system, such as Peltier plate or cooling pipes. But this prior art reference does not teach how to control cooling, nor is it concerned with the problems associated with cooling as related to virus inactivation, product preservation, etc.

### BRIEF DESCRIPTION

A first aspect of the invention relates to a bioreactor system (100) comprises a platform (102) connected to a base (112), the platform configured to rock about a pivot point (104), wherein the platform (102) has a top surface (116) configured to receive an enclosure (120); a temperature regulating element (114) attached to the platform (102), the temperature regulating element (114) include at least one channel therein; and a temperature control unit (212) fluidically connected to the at least one channel (413) and configured to heat and/or cool a gas or fluid, wherein the gas or fluid is configured to travel through the at least one channel and impart heat transfer to or from the enclosure (120) when the enclosure (120) is placed on the platform (102) and in contact with the temperature regulating element (114).

In embodiments, the temperature regulating element is configured as a plate (411) comprising: the at least one channel (413) fluidically connected to an input port (415) and an output port (417), wherein the temperature control unit (212) is configured to pass the heated or cooled gas or fluid into the input port (415), through the at least one channel (413), and out the output port (417). The plate (411) has a flat, planar top and bottom surface, and wherein the at least one channel is located within an interior of the plate (411). The at least one channel (413) occupies approximately 50-95% of a cross-sectional area of the plate (411).

In embodiments, the at least one channel (413) includes at least one protuberance (419), such that when the gas or fluid is passed through the at least one channel (413) a turbulent flow is created.

In embodiments, the at least one channel (413) has a circular, semi-circular, rectangular, or triangular cross-sectional shape, and one specific embodiment the the at least one channel (413) has a semi-circular cross-sectional shape.

In further embodiments, the temperature regulating element comprises at least one tube or pipe (716), the at least one tube or pipe (716) including an input port (717) and an output ort (718) for fluid connection with the temperature regulating unit (212).

In additional embodiments, the temperature regulating element comprises a heating/cooling blanket (726), the heating/cooling blanket (726) having at least one fluid channel therethrough and an input port (727) and an output ort (728) for fluid connection with the temperature regulating unit (212), wherein the heating/cooling blanket (724) is configured to be wrapped around the enclosure (120).

In embodiments, the enclosure is a flexible bioreactor bag.

A second aspect of the invention relates to a method (800) of heating or cooling an enclosure (120), comprising: providing a platform (102) connected to a base (112), the platform configured to rock about a pivot point (104), wherein the platform (102) has a top surface (116) configured to receive the enclosure (120); providing a temperature regulating element (114) attached to the platform (102), the temperature regulating element (114) include at least one channel (413) therein; and fluidically connecting a temperature control unit (212) to the at least one channel (413); heating or cool a gas or fluid with the temperature control unit (212); and causing the gas or fluid to travel through the at least one channel and impart heat transfer to or from the enclosure (120) when the enclsoure (120) is placed on the platform (102) and in contact with the temperature regulating element (114).

In embodiments, the temperature regulating element is a plate (411) comprising: the at least one channel (413) fluidically connected to an input port (415) and an output port (417), wherein the temperature control unit (212) passes the heated or cooled gas or fluid into the input port (415), through the at least one channel (413), and out the output port (417). The plate (411) has flat, planar top and bottom surfaces, and wherein the at least one channel is located within an interior of the plate (411).

In further embodiments, the temperature regulating element comprises at least one tube or pipe (716), the at least one tube or pipe (716) including an input port (717) and an output ort (718) for fluid connection with the temperature regulating unit (212).

In additional embodiments, the temperature regulating element comprises a heating/cooling blanket (724), the heating/cooling blanket (724) having at least one fluid channel therethrough and an input port (727) and an output ort (728) for fluid connection with the temperature regulating unit (212), wherein the heating/cooling blanket (724) is wrapped around the flexible bioreactor bag (120).

In embodiments, the at least one channel (413) includes at least one protuberance (419), such that when the gas or fluid is passed through the at least one channel (413) a turbulent flow is created.

In embodiments, the enclosure (120) is a flexible bioreactor bag.

### BRIEF DESCRIPTION OF THE DRAWINGS

FIG. 1 is an exploded view of a bioreactor system, according to embodiments of the invention.

FIG. 2 is a perspective view of a portion of the bioreactor system of FIG. 1.

FIG. 3 is a schematic diagram of a control system of a bioreactor system, according to embodiments of the invention.

FIGS. 4A-C are illustrations of a temperature regulating element and portions thereof of the bioreactor system, in accordance with embodiments of the invention.

FIGS. 5A-D illustrate thermal analysis profiles of different channel profiles of the temperature regulating element of FIGS. 4A-4C.

FIG. 6 is an illustration of a cross section of a portion of a bioreactor system, in accordance with embodiments of the invention.

FIG. 7 illustrates a perspective view and components thereof of a bioreactor system, in accordance with further embodiments of the invention.

FIGS. 8A and 8B illustrate a perspective views and components thereof of a bioreactor system, in accordance with further embodiments of the invention.

FIG. 9 illustrates a flowchart of a method for controlling temperature of a cell culture or media, implementing the bioreactor system of FIGS. 1-8.

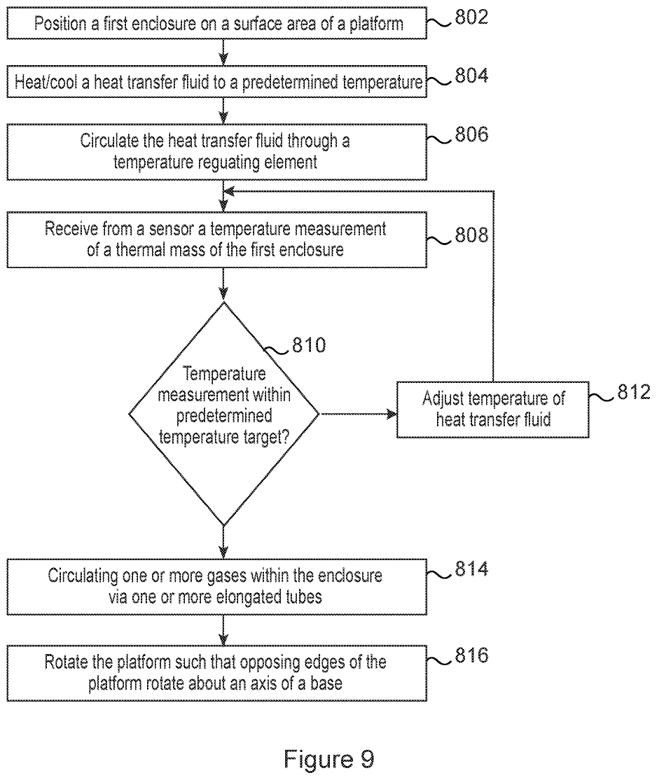

### DETAILED DESCRIPTION

Various embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors, controllers or memories) may be implemented in a single piece of hardware (e.g., a general purpose signal processor or random access memory, hard disk, or the like) or multiple pieces of hardware. Similarly, any programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Generally, various embodiments provide methods and systems for regulating temperature in a cell culture or culture media. The selected cells from a source material (e.g., T cells from blood) may be combined with a culture medium or media, which promotes cell growth. The culture medium may include Human AB serum, penicillin-streptomycin, Glutamax, Interleukin 2, NAC, and/or the like. The cell culture may be contained within an enclosure (e.g., a single-use, flexible bioreactor bag), which is also inflated with a gas mixture. The gas mixture may include nitrogen, oxygen, carbon dioxide, and/or the like, which interacts with the cell culture, for example, affecting a pH level of the cell culture. The enclosure may be made from a polymeric material, such as a plastic film or laminate, which allows for heat transfer between the cell culture and the exterior surface of the enclosure.

The enclosure may be positioned on top of a temperature regulating element contained within the bioreactor system. The temperature regulating element includes at least one input and output port connected by a channel integrated into the temperature regulating element. A fluid having a high thermal capacity such as water may be circulated through the channel by means of the input and output port, as described in more detail below.

Additionally, a cover may be positioned atop of the enclosure and is overlaid on a top portion of the enclosure adjacent to the gas mixture.

At least one technical effect of various embodiments allows for better control of temperature, with a wider temperature range, for the cell culture without making substantial hardware and/or software modifications to a conventional bioreactor. Additionally, the various embodiments provide an integrated heating and/or cooling mechanism that provides for more efficient heating and/or cooling without the need to move or transfer the cell culture or media. Specifically, the implementation of the below described temperature control unit allows better control of heating/cooling rates as compared to traditional heater pads

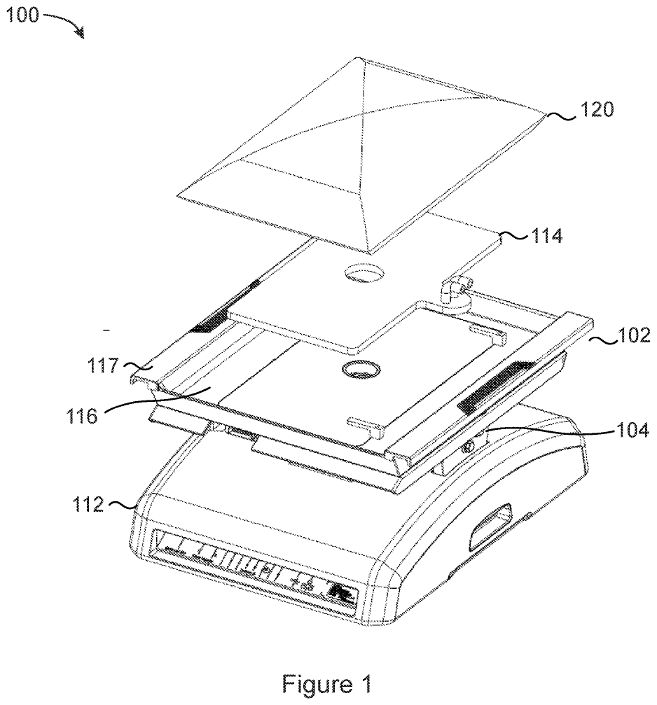

FIG. 1 is a perspective illustration of a bioreactor system 100. Optionally, a portion of the bioreactor system 100 may be similar to the WAVE Bioreactor™ Systems by Cytiva™. The bioreactor system 100 includes a platform 102. The platform 102 may include a surface area 116 surrounded by raised edges 117. The bioreactor system 100 also includes a temperature regulating element 114. The raised edges 117 may aid in securing an enclosure (e.g., enclosure 120 of FIG. 1) positioned on the surface area 116 of the platform 102. The platform 102 may be pivotably mounted on a base 112 using a pivot or pivot point 104 allowing the platform to rock back and forth across the pivot point 104. For example, the opposing edges of the platform 102 may adversely rotate about an axis corresponding to pivot point 104 of the base 112.

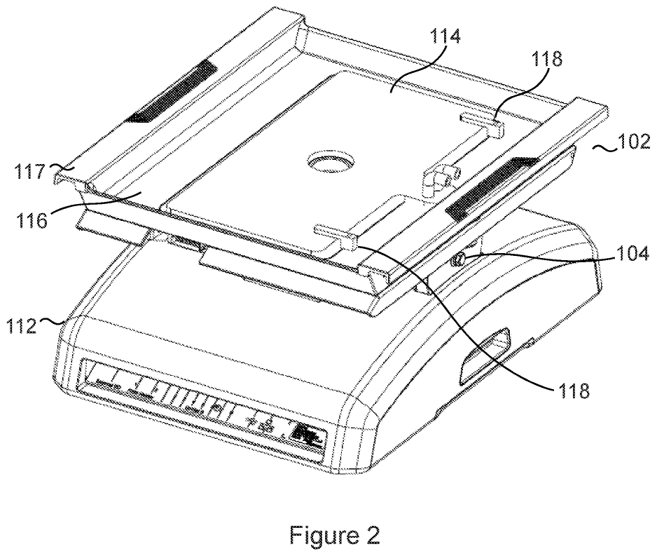

FIG. 2 is a perspective illustration of bioreactor system 100 with the temperature regulating element 114 installed and with the enclosure 120 omitted. As illustrated, temperature regulating element 114 is configured to sit within a cavity of the platform 102, thereby providing a flat surface onto which the enclosure 120 can sit. The platform 102 can also include one or more securing elements 118 configured to secure the temperature regulating element 114 to the platform 102. For example, the securing elements can be mechanical (e.g., latches, snaps), magnetic (e.g., one or more magnets that attract the temperature regulating element 114 or magnets located in or on the temperature regulating element 114), or the like. Alternatively, the temperature regulating element 114 can be shaped such that it can press fit into the cavity or can include protrusions that fit into corresponding holes within the platform 102.

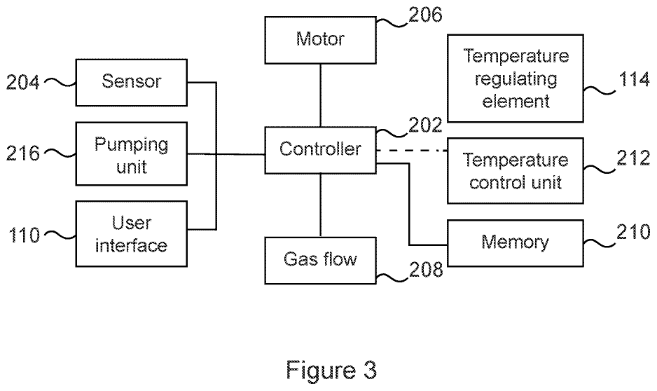

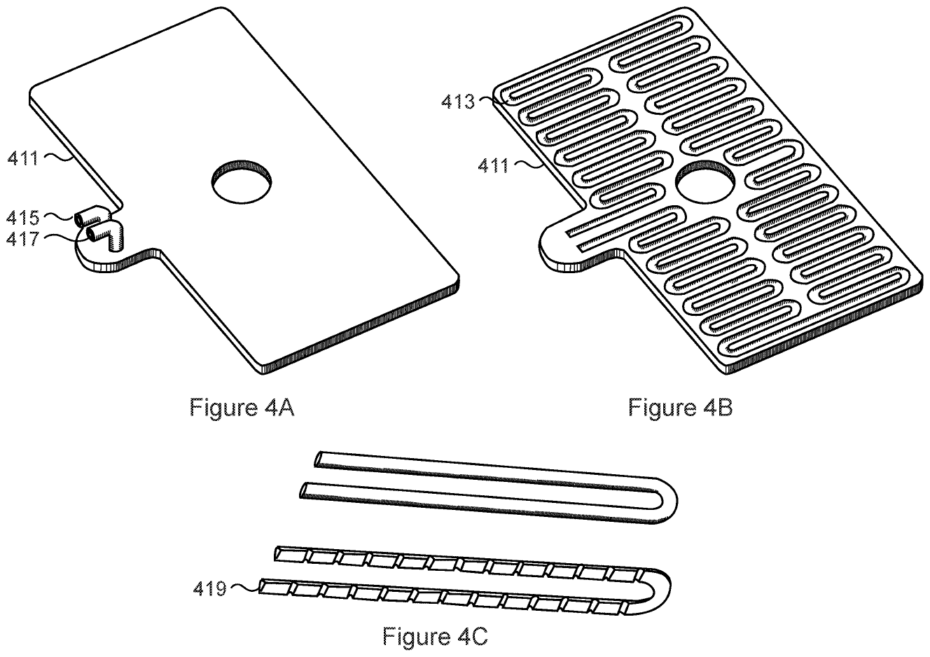

The base 112 may enclose electrical and/or control components, such as the components shown in FIG. 3 of the bioreactor system 100. Alternatively, the components of FIG. 3 can be provided in a separate device that is in communication (e.g., electrical and fluid communication) with the bioreactor system 100. As will be described in greater detail with regard to FIGS. 4A-C, temperature regulating element 114 includes at least one channel 413 that is configured to allow flow of a fluid therethrough to effectuate heating and/or cooling of the enclosure 120 when the enclosure 120 is placed on top of temperature regulating element 114 and in the bioreactor system 100.

FIG. 3 is a schematic block diagram 200 of the bioreactor system 100. The controller circuit 202 (hereafter referred to as controller or controller circuit, interchangeably) may include one or more processors, a central processing unit (CPU), a microprocessor, and/or any other electronic component capable of processing inputted data according to a specific logical instruction. For example, the controller circuit 202 may execute program instructions that are stored on memory 210 to perform one or more programmed operations. The memory 210 may include RAM, ROM, EEPROM, and/or other tangible and non-transitory computer readable medium. Additionally or alternatively, the memory 210 may be integrated with the controller circuit 202. The controller circuit 202 may be electrically and/or communicatively coupled to a sensor 204, a motor 206, a user interface 110, gas flow interface 208, the memory 210, and a pumping unit 216. Additionally, a temperature control unit (TCU) 212 may be included in the bioreactor system 100 and connected to the temperature regulating element 114. The TCU 212 can include its own control circuity or be controlled by and electrically and/or communicatively coupled controller circuit 202.

The motor 206 may be configured to control and/or adjust a position of the platform 102 with respect to the base 112 based on instructions received by the controller circuit 202. The motor 206 may be an electric motor, an actuator, and/or other electromechanical device. The motor 206 may control a rocking speed and angle of the platform 102 for agitating (e.g., displacing) a cell culture positioned on the platform 102. The rocking speed may correspond to a rate at which the opposing edges move. The angle of the platform 102 may correspond to a maximum angular or distance an opposing edge may travel, respectively, before changing direction. For example, the rocking speed may be two rocks per minute at an angle of two degrees relative to a horizontal plane. It should be noted that in other embodiments the rocks per minute and angle may be greater than and/or less than two, respectively.

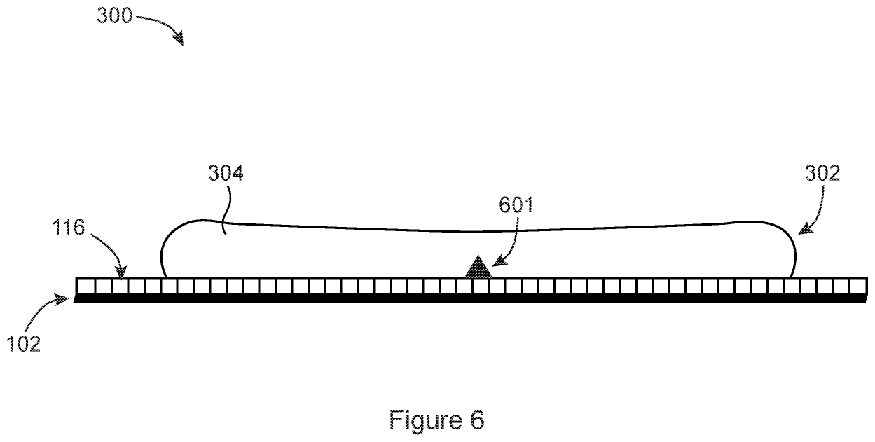

The sensor 204 may be a temperature sensor such as a thermocouple, thermistor, and/or the like. The sensor 204 may be positioned and/or configured to measure a temperature of a fluid (e.g., cell culture or media 304 located within enclosure 120 of FIG. 6) on top of the platform 102. For example, the sensor 204 may be positioned proximate to and/or in contact with the enclosure 120. Temperature measurements of the sensor 204 may be received by the controller 202 and/or compared by the controller 202 to a predetermined temperature target.

For example, during a cell culture process, the predetermined temperature target may be 37.5 degrees Celsius. It should be noted in other embodiments, the predetermined temperature target may be greater than or less than 37.5 degrees Celsius. In another embodiment, the predetermined temperature target may be a range about a set point. For example, the predetermined temperature target may be a 0.2 degree Celsius range about 37.5 degrees Celsius. Similarly, when cooling is desired, a predetermined temperature target or target range, such as 2-8 degrees Celsius can be set. In one preferred embodiment, the predetermined temperature is 4 degrees Celsius.

Based on the temperature measurements of the sensor 204 with respect to the predetermined temperature target, the controller 202 or TCU 212 may adjust an amount of heating and/or cooling at the temperature regulating element 114, which will be discussed in greater detail below.

The user interface 110 may include a keypad, a display, a keyboard, a touchscreen, tactile buttons, and/or the like for sending various instructions to the controller circuit 202. For example, the controller circuit 202 may receive instructions to increase the rocks per minute of the platform based on instructions received from the user interface 110. The user interface 110 may be positioned on an outer surface of the base 112. Additionally or alternatively, the user interface 110 may be positioned on the platform and/or remote from the base 112 (e.g., a computer communicatively coupled to the bioreactor system 100).

The gas flow interface 208 may be configured to control a flow rate of one or more gases carried by a plurality of elongated tubes (not shown) from one or more tanks or containers (not shown) to an enclosure (e.g., enclosure 120), which is absorbed by the cell culture. The gas flow interface 208 may be a flow limiter, a mass flow controller, a gas pump and/or the like. The one or more tanks may supply one or more gases, which are carried by the elongated tubes. For example, one or more tanks may contain one or more gases, such as nitrogen, oxygen, carbon dioxide, and/or the like, which are delivered or carried by the elongated tubes. Optionally, the gas flow interface 208 may combine and/or mix gases from one or more tanks into a gas mixture, which is carried by one or more of the elongated tubes.

The gas flow interface 208 may receive instructions from the controller circuit 202 to regulate an amount of gas within an enclosure coupled to the elongated tubes. For example, the elongated tubes may deliver a gas mixture of oxygen, carbon dioxide and/or nitrogen into an enclosure (e.g., enclosure 120), and may exhaust the gas mixture from the enclosure to circulate the gas mixture within the enclosure.

In various embodiments, the gas flow interface 208 may control a gas flow rate and/or circulation of the gas mixture within the enclosure (e.g., enclosure 120) based on instructions received by the controller 202. For example, the delivery and exhaust of the gas mixture via the elongated tubes, respectively, may displace portions of the gas mixture by circulating the gas mixture or move the gas mixture within the enclosure between the elongated tubes. The controller 202 may instruct the gas flow interface 208 reach a gas flow rate within the enclosure based on a gas requirement of the cell culture.

Optionally, the gas flow rate (e.g., rate of gas traversing through the elongated tubes) may be based on a volume of the cell culture or media within the enclosure. For example, the controller 202 may have the gas flow rate be lower (e.g., 0.02 L/min) for enclosures with cell cultures having a lower volume relative to the gas flow rate (e.g., 0.1 L/min) of enclosures having higher volume cell cultures.

It should be noted in other embodiments the bioreactor system 100 may include more than two tubes (e.g., five tubes), and the pumping unit 216 may be coupled to one or more tubes (not shown) in contact with the cell culture.

The pumping unit 216 may be configured to move fluid into and out of an enclosure (e.g., enclosure 120). For example, the pumping unit 216 may feed media and/or the cell culture from a tank carried by an elongated tube into the enclosure 120. In another example, the pumping unit 216 may remove waste media from the cell culture carried by an elongated tube to a waste tank. The pumping unit 216 may receive instructions from the controller 202, which determines when to add and/or remove fluid from the enclosure. The pumping unit 216 may be a displacement pump that includes a cavity to create a suction for moving fluid within the pumping unit 216 from a source location (e.g., tank, enclosure) to a discharge location (e.g., enclosure, waste tank). Optionally, the pumping unit 216 may include one or more rotors and/or plungers to move fluid within the pumping unit 216.

In connection with FIGS. 4A-C, the temperature regulating element 114 is configured to heat and/or cool the surface area 116 of the platform 102. According to an embodiment, the temperature regulating element 114 is in the form of a plate 411 having at least one channel 413 integrated therein. The channel is connected to an input port 415 and an output port 417. As illustrated in FIG. 4A, the top of the plate 411 is solid. However, FIG. 4B illustrates a cross section of the plate, showing an internal, serpentine channel 413 fluidically connected to the input and output ports 415, 417. Said another way, the plate 411 has a flat, planar top and bottom surface, and wherein the at least one channel is located within an interior of the plate 411. In this way, a heated or cooled fluid or gas can be passed through the input port 415, through the channel 413, and out of the output port 417. The TCU 212 (or the controller 202) is configured to control the temperature of the gas or fluid and circulate said gas or fluid through the channel when heating or cooling is desired. An additional benefit of such a design is that the plate 411 may be modular, and thus added or removed from the bioreactor system 100 easily and without the need to make modifications to the bioreactor system 100.

The channel 413, while illustrated as a serpentine, does not need to take the exact shape as shown in the Figures. For example, the channel can have ninety degree turns, turn about the length of the plate 411 (as compared to its width), etc. In embodiments, the channel covers up to 95% of the cross-sectional area of the plate 411. The channel can also cover varying amounts of the area of the plate, such as 85-95%, 75-95%, 65-95%, 50-95%, and all ranges therebetween. It is desirable that the channel cover a substantial portion of the area of the plate 411 to ensure efficient heat transfer with the cell culture or media 304. Additionally, as illustrated by FIG. 4C, the channel 413 may have a uniform shape (top of FIG. 4A) or include protuberances 419 (e.g., bumps). The protuberances 419 can aid in creating turbulent fluid flow through the channel 413, which provides more efficient heat transfer.

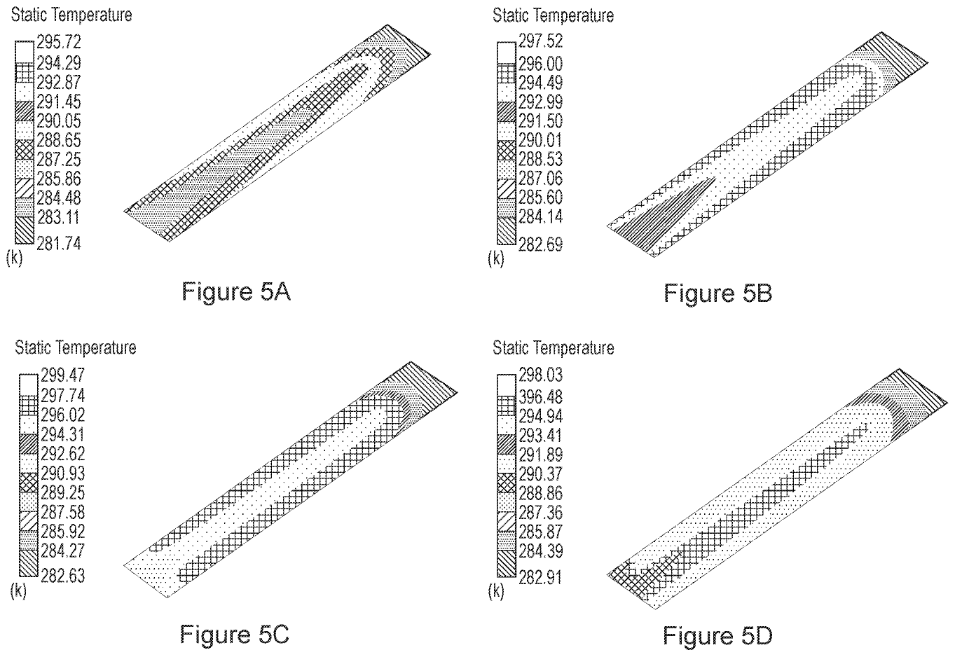

The cross-sectional area of the channel 413 can also take a variety of shapes, including circular, semi-circular, rectangular, triangular, honeycomb, etc. FIGS. 5A-D illustrate thermal modeling of different cross-sectional shapes. FIG. 5A shows the thermal profile for a circular cross section (e.g., traditional pipe shape), FIG. 5B shows the thermal profile for a semi-circular cross section with a flat bottom, FIG. 5C shows the thermal profile for a semi-circular cross section including protuberances 419 along the bottom, and FIG. 5D shows the thermal profile for a triangular cross section. As can be seen, the semi-circular cross section with including protuberances 419 has the best heat transfer, and is thus a preferred cross-sectional shape for the channel, although other shapes are within the scope of the invention.

In order to manufacture the temperature regulating element 114 a variety of techniques can be employed. For example, the temperature regulating element 114 can be made using traditional machining techniques. In a preferred embodiment, the temperature regulating element 114 can be manufactured using additive manufacturing techniques (e.g., 3D printed). The material selected for the temperature regulating element 114 can include, but is not limited to metals, such as stainless steel, aluminum, or copper.

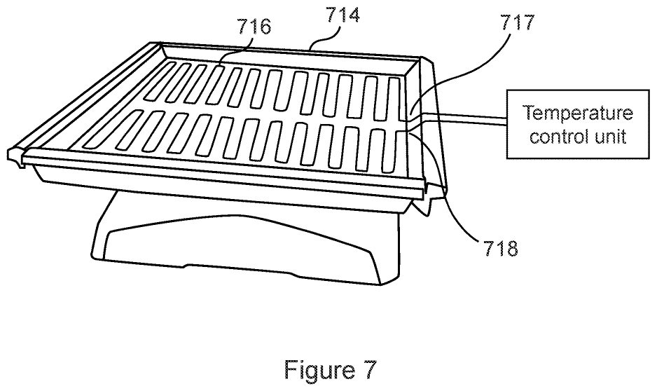

FIG. 7 illustrates an alternative embodiment for heating and/or cooling the enclosure 120. As shown, the temperature regulating element 714 can take the form of tubing or piping 716. Similar to the embodiment shown in FIG. 1, the temperature regulating element has input and output ports 717, 718 connected to TCU 212 through additional tubing or pipes. The tubing or piping 716 is located on platform 102 and can have a serpentine shape such that it covers up to 95% of the area of the platform 102. The tubing or piping 716 can also cover varying amounts of the area of the platform 102, such as 85-95%, 75-95%, 65-95%, 50-95%, and all ranges therebetween. It is desirable that the tubing or piping 716 cover a substantial portion of the area of platform 102 to ensure efficient heat transfer with the thermal mass 304.

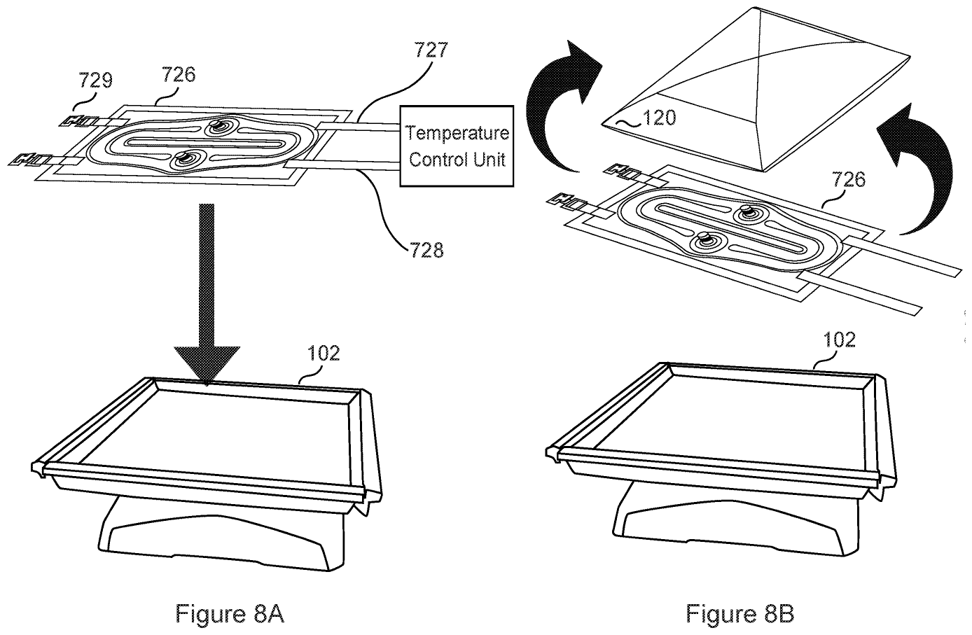

FIG. 8Aand 8B illustrate a further alternative embodiment for heating and/or cooling the enclosure 120. As shown, the temperature regulating element 726 can take the form of a heating/cooling blanket 726. The heating/cooling blanket 726 is located on platform 102 and includes a fluid channel through its interior. Similar to the embodiment shown in FIG. 1, the heating/cooling blanket 726 has input and output ports 727, 728 connected to TCU 212 through a fluid connection. The heating/cooling blanket 726, unlike other embodiments, can be wrapped around and attached to the first enclosure 120 (e.g., by way of fasteners 729), such that heating/cooling can be imparted on a larger surface area of the thermal mass 304.

With reference to the above embodiments, the TCU 212 includes a source of heat transfer fluid (e.g., water). The heat transfer fluid is fluidically connected to the channel 413 (or tubing or piping 716) of the temperature regulating element 114, 714 via tubing connect to the input and output ports 415, 417, 717, 718. The TCU 212 includes heating and cooling elements configured to heat and cool the heat transfer fluid to set temperatures. In addition, a pump (not shown) is controlled by the controller circuit 202 or TCU 212 to pump the heat transfer fluid through the tubes, into the input port 415, 717 and out of the output port 417, 718 thereby circulating the heat transfer fluid through the channel 413 or tubing or piping 716. When the heat transfer fluid is cooled, the cold fluid draws heat energy out of the cell culture or media 304 when it is placed on the temperature regulating element 114 (see, e.g., FIG. 6). Similarly, when the heat transfer fluid is heated, the warm fluid transfers heat to the cell culture or media 304 when the enclosure 120 is placed on the temperature regulating element 114.

The controller circuit 202 or TCU 212 may adjust the temperature of the heat transfer fluid based on measurements by the temperature sensor 204. For example, the controller circuit 202 or TCU 212 may be instructed to have the temperature of the cell culture or media 304 reach a predetermined set point (e.g., 2 °C for cooling or 37.5 °C for heating).

FIG. 6 is a cross section 300 of the platform 102 with a first enclosure 302. The first enclosure 302 may be a flexible container or bag, and is composed of a plastic material. For example, the first enclosure 302 may be formed from layers of polyvinyl chloride and ethyl vinyl acetate. The cross section 300 illustrates the temperature regulating element 114 in contact with the first enclosure 302 and located on the platform 102. As the heat transfer fluid is circulated through the channel 413 the surface area 116, as well as the top surface of plate 411, is heated or cooled. In this way, heat is absorbed by first enclosure 302 or withdrawn from the first enclosure 302.

FIG. 6 also illustrates an optional baffle 601. The baffle 601 is in the form of a long protuberance that can either be integrated into the surface area 116 or the enclosure 302 and should be of a sufficient length to span across the entire width of the enclosure 302. By including baffle 601 turbulence of the fluid within the enclosure 302 is increased when rocking occurs. This increased turbulence both aids in mixing of the contents of the enclosure as well as aiding in heat transfer between the temperature regulating element 114 and the thermal mass 304.

FIG. 8 illustrates a flowchart of a method for controlling temperature of a cell culture or media. The method 800, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 800 may be used as one or more algorithms to direct hardware to perform one or more operations described herein. It should be noted, other methods may be used, in accordance with embodiments herein.

One or more methods may include (i) positioning a first enclosure on a surface area of a platform, (ii) heating or cooling a heat transfer fluid, (iii) circulate the heat transfer fluid through a temperature regulating element such that the surface area of the platform is heated or cooled, thereby imparting or removing heat from the first enclosure.

Beginning at 802, the first enclosure 120, 302 containing a cell culture or media 304, is positioned on the surface area 116 of the platform 102. For example, the first enclosure 120, 302 is placed adjacent to the surface area 116 of the platform 102. As FIGS. 2 and 6 illustrate, the temperature regulating element 114 is also located on the platform 102 such that when the enclosure 120, 302 is placed on the platform 102 it is thermally coupled to the cell culture or media 304.

At 804, depending upon the desired result, a heat transfer fluid is heated or cooled to a predetermined temperature. For example, according to embodiments, the bioreactor system 100 includes a reservoir of the heat transfer fluid as well as at least one heating element and at least one cooling element in communication with the fluid reservoir. Based on a preset or user-defined temperature value the controller 202 or TCU 212 heats or cools the fluid to the value.

At 806, the heat transfer fluid is circulated through the channel 413 of the temperature regulating element 114, 714 (e.g., using a pump).

At 808, heating or cooling is generated on the surface area 116 of the platform 102, at the location of the surface of the temperature regulating element 114, 714. The amount of heating or cooling may be determined and/or controlled by the controller 202 or TCU 212. For example, the controller 202 may control an amount of current and/or voltage to a heating element or cooling element which is used to heat or cool the heat transfer fluid.

At 810, the controller 202 or TCU 212 may receive temperature measurements of the cell culture or media 304 of the first enclosure 120, 302 from the sensor 204. For example, the sensor 204 may be positioned proximate and/or adjacent to the first enclosure 120, 302. The cell culture or media 304 absorbs at least some of the heat (or is cooled) resulting in a temperature change of the cell culture or media 304. The sensor 204 may acquire temperature measurements corresponding to a temperature of the cell culture or media 304, which are received by the controller 202 TCU 212.

At 812, the controller 202 or TCU 212 may determine whether the temperature measurement is within a predetermined temperature target. The predetermined temperature target may be a preset or user-defined temperature value and be stored on the memory 210. Optionally, the predetermined temperature target may be received by the controller 202 from the user interface 110. The predetermined temperature target may correspond to a temperature approximate to a desired temperature for culturing the cell culture or media 304, a temperature for viral inactivation, or a temperature for cold storage, or a temperature for another desired application. The controller 202 may compare the temperature measurement acquired by the sensor 204 to the predetermined temperature target. Based on a difference between a value of the temperature measurement and the predetermined temperature target, the controller 202 can determine whether the cell culture or media 304 is at the desired temperature.

For example, the predetermined temperature target may be set at 37.5 degrees Celsius (for cell culture) or 2 degrees Celsius (for virus inactivation or cold storage). The controller 202 or TCU 212 may determine that temperature measurements not within 0.2 degrees of the predetermined temperature target, such as greater than 37.9 (or 2.2) degrees Celsius or less than 37.3 (or 1.8) degrees Celsius are determined not to be within the predetermined temperature target.

If the temperature measurement is determined by the controller 202 or TCU 212 to not be within the predetermined temperature target, then at 812, the controller 202 or TCU 212 adjusts the temperature of the heat transfer fluid. For example, if the temperature measurement from the sensor 204 is below the predetermined temperature target, the controller 202 may provide additional heating to the heat transfer fluid. In another example, if the temperature measurement from the sensor 204 is above the predetermined temperature target, the controller 202 or TCU 212 further cools the heat transfer fluid.

In a further optional step, if the temperature measurement is determined by the controller 202 to be within the predetermined temperature target, then at 814, one or more gases are circulated within the enclosure 120, 302 via the one or more elongated tubes. For example, the controller 202 may instruct the gas flow interface 208 to inject one or more gases into the enclosure 120, 302, and exhaust one or more gases from the enclosure 120, 302 via the elongated tubes, respectively. The one or more gases may include nitrogen, oxygen, carbon dioxide, and/or the like. The controller 202 may determine the proportion of gases injected and/or exhausted from the enclosure 120, 302 based on a predetermined setting stored on the memory 210. Additionally or alternatively, the proportion of gases may be received by the controller 202 based on inputs received by the user interface 110.

In a further optional step, at 816, the platform 102 is rotated such that opposing edges 122 and 124 of the platform 102 rotate about the axis 120 of the base 112. For example, the controller 202 may enable and/or instruct the motor 206 to rock back and forth across the pivot point 104. A rocking speed and angle of the platform 102 during rotation may be determined by the controller 202 from predetermined settings store on the memory 210. Additionally or alternatively, the rocking speed and angle of the platform 102 may be received by the controller 202 based on inputs received by the user interface 110.

It should be noted that the particular arrangement of components (e.g., the number, types, placement, or the like) of the illustrated embodiments may be modified in various alternate embodiments. For example, in various embodiments, different numbers of a given module or unit may be employed, a different type or types of a given module or unit may be employed, a number of modules or units (or aspects thereof) may be combined, a given module or unit may be divided into plural modules (or sub-modules) or units (or sub-units), one or more aspects of one or more modules may be shared between modules, a given module or unit may be added, or a given module or unit may be omitted.

As used herein, a structure, limitation, or element that is “configured to” perform a task or operation may be particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a processing unit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid state drive, optic drive, and the like. The storage device may also be other similar means for loading computer programs or other instructions into the computer or processor.

As used herein, the term “computer,” “controller,” and “module” may each include any processor-based or microprocessor-based system including systems using microcontrollers, reduced instruction set computers (RISC), application specific integrated circuits (ASICs), logic circuits, GPUs, FPGAs, and any other circuit or processor capable of executing the functions described herein. The above examples are exemplary only, and are thus not intended to limit in any way the definition and/or meaning of the term “module” or “computer.”

The computer, module, or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer, module, or processor as a processing machine to perform specific operations such as the methods and processes of the various embodiments described and/or illustrated herein. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program. The individual components of the various embodiments may be virtualized and hosted by a cloud type computational environment, for example to allow for dynamic allocation of computational power, without requiring the user concerning the location, configuration, and/or specific hardware of the computer system.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f) unless and until such claim limitations expressly use the phrase “means for” followed by a statement of function void of further structure.

This written description uses examples to disclose the various embodiments, and also to enable a person having ordinary skill in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal languages of the claims.

The foregoing description of certain embodiments of the present inventive subject matter will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (for example, processors or memories) may be implemented in a single piece of hardware (for example, a general purpose signal processor, microcontroller, random access memory, hard disk, or the like). Similarly, the programs may be stand alone programs, may be incorporated as subroutines in an operating system, may be functions in an installed software package, or the like. The various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “comprises,” “including,” “includes,” “having,” or “has” an element or a plurality of elements having a particular property may include additional such elements not having that property.

What is claimed is:

1. A bioreactor system comprising:
a platform connected to a base, the platform configured to rock about a pivot point wherein the platform has a top surface configured to receive an enclosure
a temperature regulating element attached to the platform the temperature regulating element including at least one channel therein; and
a temperature control unit fluidically connected to the at least one channel and configured to heat and/or cool a gas or fluid, wherein the gas or fluid is configured to travel through the at least one channel and impart heat transfer to or from the enclosure when the enclosure is placed on the platform and in contact with the temperature regulating element.

2. The bioreactor system of claim 1, wherein the temperature regulating element is a plate comprising:
the at least one channel fluidically connected to an input port and an output port
wherein the temperature control unit is configured to pass the heated or cooled gas or fluid into the input port, through the at least one channel, and out the output port.

3. The bioreactor system of claim 3, wherein the plate has a flat, planar top and bottom surface, and wherein the at least one channel is located within an interior of the plate.

4. The bioreactor system of claim 2, wherein the at least one channel occupies approximately 50-95% of a cross-sectional area of the plate.

5. The bioreactor system of claim 1, wherein the at least one channel includes at least one protuberance, such that when the gas or fluid is passed through the at least one channel a turbulent flow is created.

6. The bioreactor system of claim 1, wherein the at least one channel has a circular, semi-circular, rectangular, or triangular cross-sectional shape.

7. The bioreactor system of claim 1, wherein the at least one channel has a semi-circular cross-sectional shape.

8. The bioreactor system of claim 1, wherein the temperature regulating element comprises at least one tube or pipe, the at least one tube or pipe including an input port and an output ort for fluid connection with the temperature regulating unit.

9. The bioreactor system of claim 1, wherein the temperature regulating element comprises a heating/cooling blanket, the heating/cooling blanket having at least one fluid channel therethrough and an input port and an output ort for fluid connection with the temperature regulating unit, wherein the heating/cooling blanket is configured to be wrapped around the enclosure.

10. The bioreactor system of claim 1, wherein the enclosure is a flexible bioreactor bag.

11. A method of heating or cooling an enclosure, comprising:
providing a platform connected to a base, the platform configured to rock about a pivot point, wherein the platform has a top surface configured to receive the enclosure;
providing a temperature regulating element attached to the platform, the temperature regulating element include at least one channel therein; and
fluidically connecting a temperature control unit to the at least one channel;
heating or cool a gas or fluid with the temperature control unit; and
causing the gas or fluid to travel through the at least one channel and impart heat transfer to or from the enclosure when the enclosure is placed on the platform and in contact with the temperature regulating element.

12. The method of claim 11, wherein the temperature regulating element is a plate comprising:
the at least one channel fluidically connected to an input port and an output port,
wherein the temperature control unit passes the heated or cooled gas or fluid into the input port, through the at least one channel, and out the output port.

13. The method of claim 12, wherein the plate has flat, planar top and bottom surfaces, and wherein the at least one channel is located within an interior of the plate.

14. The method of claim 11, wherein the temperature regulating element comprises at least one tube or pipe, the at least one tube or pipe including an input port and an output ort for fluid connection with the temperature regulating unit.

15. The method of claim 11, wherein the temperature regulating element comprises a heating/cooling blanket, the heating/cooling blanket having at least one fluid channel therethrough and an input port and an output ort for fluid connection with the temperature regulating unit, wherein the heating/cooling blanket is wrapped around the flexible bioreactor bag.

16. The method of claim 11, wherein the at least one channel includes at least one protuberance, such that when the gas or fluid is passed through the at least one channel a turbulent flow is created.

17. The method of claim 11, wherein the enclosure is a flexible bioreactor bag.
